(19) United States
(12) Patent Application Publication
(10) Pub. No.: US 2026/0290332 A1
(43) Pub. Date: Sep. 24, 2026

(54) CONVERSION OF LOW AMPLITUDE VOICE DETECTION TO TEXT

(71) Applicant: Augmental Technologies Inc., San Francisco, CA (US)

(72) Inventors: Tomas Alfonso Vega Galvez, San Francisco, CA (US); Suraj Raju Gowda, Walnut Creek, CA (US)

(73) Assignee: Augmental Technologies Inc., San Francisco, CA (US)

(21) Appl. No.: 19/696,443

(22) Filed: Jun. 3, 2026

Related U.S. Application Data
(63) Continuation-in-part of application No. 19/459,021, filed on Jan. 26, 2026.
Application No. 19/459,021 is a continuation-in-part of application No. 18/888,237, filed on Sep. 18, 2024.
Application No. 18/888,237 is a continuation-in-part of application No. 18/099,288, filed on Jan. 20, 2023.
Application No. 18/099,288 is a continuation-in-part of application No. 17/366,186, filed on Jul. 2, 2021, now Pat. No. 11,941,161.
(60) Provisional application No. 63/047,946, filed on Jul. 3, 2020.
(60) Provisional application No. 63/063,455, filed on Aug. 10, 2020.
(60) Provisional application No. 63/162,444, filed on Mar. 17, 2021.
(60) Provisional application No. 63/301,501, filed on Jan. 21, 2022.
(60) Provisional application No. 63/539,130, filed on Sep. 19, 2023.
(60) Provisional application No. 63/749,766, filed on Jan. 27, 2025.
(60) Provisional application No. 63/817,481, filed on Jun. 4, 2025.
(60) Provisional application No. 63/940,179, filed on Dec. 13, 2025.
(60) Provisional application No. 63/963,061, filed on Jan. 19, 2026.

(51) Int. Cl. G10L 15/183; G10L 15/25; G10L 21/0208; H04R 1/46; H04R 17/02

(52) U.S. Cl. CPC G10L 15/183; G10L 15/25; G10L 21/0208; H04R 1/46; H04R 17/02

(57) ABSTRACT

An acoustic horn is accessed. The acoustic horn is coupled to a head of a user. Audio from a whispered voice of the user is obtained. The whispered voice includes one or more words. The words can comprise a full natural language input. A signal processing module generates audio data. The audio data is based on the whispered voice that was obtained. The audio data is translated, by an automatic speech recognition (ASR) model, into text. The text corresponds to the one or more words. The ASR model can send the text to a large language model (LLM). The acoustic horn transmits sound energy from the user to the signal processing module. The acoustic horn includes one or more impedance matching layers.

### RELATED APPLICATIONS

This application claims the benefit of U.S. provisional patent applications “Conversion Of Low Amplitude Voice Detection To Text” Ser. No. 63/817,481, filed Jun. 4, 2025, “Listening Device For Whisper Speech Detection With Passive Noise Blocking” Ser. No. 63/940,179, filed Dec. 13, 2025, and “Microphone Array For Multimodal Sound Detection With A DSP Pipeline” Ser. No. 63/963,061, filed Jan. 19, 2026.

This application is also a continuation-in-part of U.S. patent application “Context-Aware Gestures With An Oral Sensor Interface” Ser. No. 19/459,021, filed Jan. 26, 2026, which claims the benefit of U.S. provisional patent applications “Context-Aware Gestures With An Oral Sensor Interface” Ser. No. 63/749,766, filed Jan. 27, 2025, and “Conversion Of Low Amplitude Voice Detection To Text” Ser. No. 63/817,481, filed Jun. 4, 2025.

The U.S. patent application “Context-Aware Gestures With An Oral Sensor Interface” Ser. No. 19/459,021, filed Jan. 26, 2026 is also a continuation-in-part of U.S. patent application “Silent Intraoral Speech Sensing With Audio Feedback” Ser. No. 18/888,237, filed Sep. 18, 2024, which claims the benefit of U.S. provisional patent application “Silent Intraoral Speech Sensing With Audio Feedback” Ser. No. 63/539,130, filed Sep. 19, 2023.

The U.S. patent application “Silent Intraoral Speech Sensing With Audio Feedback” Ser. No. 18/888,237, filed Sep. 18, 2024 is also a continuation-in-part of U.S. patent application “Intraoral Electronic Sensing for Health Monitoring” Ser. No. 18/099,288, filed Jan. 20, 2023, which claims the benefit of U.S. provisional patent application “Intraoral Electronic Sensing for Health Monitoring” Ser. No. 63/301,501, filed Jan. 21, 2022.

The U.S. patent application “Intraoral Electronic Sensing for Health Monitoring” Ser. No. 18/099,288, filed Jan. 20, 2023 is also a continuation-in-part of U.S. patent application “Data Manipulation Using Remote Augmented Sensing” Ser. No. 17/366,186, filed Jul. 2, 2021, issued as U.S. patent number Ser. No. 11/941,161, Mar. 26, 2024, which claims the benefit of U.S. provisional patent applications “Data Manipulation Using Remote Augmented Sensing” Ser. No. 63/047,946, filed Jul. 3, 2020, “Gestural Sensing Using In-Ear Inertial Measurements” Ser. No. 63/063,455, filed Aug. 10, 2020, and “Intraoral Connected Processing Devices” Ser. No. 63/162,444, filed Mar. 17, 2021.

Each of the foregoing applications is hereby incorporated by reference in its entirety.

### FIELD OF ART

This application relates generally to audio processing and more particularly to conversion of low amplitude voice detection to text.

### BACKGROUND

Communication is a basic function of any human society. Without communication, cooperation among people becomes difficult, if not impossible. Human communication can take on many forms, such as speech, gestures, body movements, grunts, nods, and so on. More advanced communication can include developing a written language system corresponding to a speech system. Even more advanced, perhaps, is some kind of direct interface with a person's brain, either by detecting brainwaves or even by direct sensing of neural paths within the brain and/or body. Such an interface can then be associated with some kind of external means to bring inner thoughts and feelings to light for others. An even more futuristic way of communicating has been described in science fiction books, detailing a form of direct mental telepathy between two or more individuals. However, for the time being, a combination of two or more communication modalities can be employed to achieve superior efficacy in the communication process. No matter how communication is performed, it is clearly a necessity for human survival.

The lack of communication ability, either temporarily or long term, can be a significant detriment to any given experience. For example, traveling to a foreign country where one's native tongue is not spoken can be a difficult and daunting experience. On the other hand, games that limit or disallow the use of verbal communication can be fun and even challenging activities. For a lighter example, consider the parlor game “charades.” Charades prohibits speech altogether and relies on bodily signs, movements, reenactments, and expressions to encourage teammates to guess the word or words being communicated. Intentionally avoiding spoken communication can not only be an enjoyable pastime, but it can also develop secondary communication skills and inculcate an appreciation of the difficulties in communication experienced by those unable to speak or hear.

The human auditory system is amazing, enabling people to hear surrounding sound energy. At a basic level, the system includes the ears, auditory nerves, and brain. Sound waves enter the outer ear, causing the eardrum to vibrate. These vibrations are transmitted to the cochlea, a spiral shaped structure in the inner ear, which includes cilia. The cilia are sensitive hair cells which convert the vibrations from the eardrum into electrical signals carried to the brain via the auditory nerve. The brain then interprets the electrical signals as sound. An undamaged human auditory system can typically process frequencies between 20Hz and 20kHz. However, age, damage, and other factors can cause this range to narrow. For example, excessive exposure to loud noises can damage the cilia, which do not regenerate. Damage can cause conditions such as tinnitus or hearing loss. Hearing range can also become limited with age, especially at higher frequencies. Modern technology, including hearing aids and cochlear implants, can assist those with auditory challenges. As technology continues to advance, those with hearing problems will be helped even further.

### SUMMARY

The rise of artificial intelligence (AI) has brought to life new technologies that previously could only be imagined. Self-driving cars, more accurate cancer detection from medical imaging, fraud detection, personalized learning, and audio/video content creation are a few examples of how AI is changing everyday lives. In particular, large language models (LLMs) are having a growing influence on the way research is performed and common questions are answered. LLMs can be accessed by any number of compute devices, including mobile devices. However, users typically interact with processing devices by typing, clicking, touching, pressing, swiping, and employing other familiar input/output techniques. Some devices include audio interfaces, which can speed up human interactions with an LLM. However, audio interfaces are not always convenient when in a crowd of people, when in a loud environment, and so on.

Disclosed techniques enable conversion of low amplitude voice detection to text. An acoustic horn is accessed. The acoustic horn is coupled to a head of a user. Audio from a whispered voice of the user is obtained. The whispered voice includes one or more words. The words can comprise a full natural language input. A signal processing module generates audio data. The audio data is based on the whispered voice that was obtained. The audio data is translated, by an automatic speech recognition (ASR) model, into text. The text corresponds to the one or more words. The ASR model can send the text to a large language model (LLM).

A processor-implemented method for data manipulation is disclosed comprising: accessing an acoustic horn, wherein the acoustic horn is coupled to a head of a user; obtaining, by the acoustic horn, audio from a whispered voice of the user, wherein the whispered voice includes one or more words; generating audio data, by a signal processing module, wherein the audio data is based on the whispered voice that was obtained; and translating, by an automatic speech recognition (ASR) model, the audio data into text, wherein the text corresponds to the one or more words. Some embodiments comprise sending, by the ASR model, the one or more words to a large language model (LLM). In embodiments, the acoustic horn transmits sound energy from the user to the signal processing module. In embodiments, the acoustic horn includes one or more impedance matching layers. In embodiments, the acoustic horn includes two microphones. In embodiments, the two microphones comprise a primary microphone and a reference microphone.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for conversion of low amplitude voice detection to text.

FIG. 2 is a flow diagram for acoustic horn constituency.

FIG. 3 is an example of placing an acoustic horn on a user.

FIG. 4 is a diagram of an acoustic horn.

FIG. 5 is an infographic of signal processing using a digital signal processor (DSP) pipeline.

FIG. 6 is a block diagram for a dual-microphone audio processor.

FIG. 7 is an example neural network.

FIG. 8 is a system diagram for conversion of low amplitude voice detection to text.

### DETAILED DESCRIPTION

Audio interfaces have proven to be a convenient way for users to interact with technology. Today, users can speak keywords to compute devices such as mobile phones, smartwatches, tablets, laptops, smart appliances, and so on to engage with a digital assistant. Functions that once required physical touch, a mouse, a keyboard, etc. can now be accomplished with a simple voice command. For those with disabilities, audio entry can provide a means to operate compute devices without a mouse, keyboard, etc. Audio can also be used as an input for machine learning models, such as LLMs to speed up interactions, research, etc. While this can be more convenient than other types of input, such as a mobile phone virtual keyboard, issues exist that limit the use of audio input to LLMs in the real world. For example, if captured audio from a user lacks a sufficient level of clarity, a machine learning model may not understand the user's input, leading to unreliable results, hallucinated responses, and so on. This can be especially true in environments with noisy backgrounds, multiple voices, and so on. Further, some users may feel self-conscious speaking into a microphone with sufficient volume to establish a reliable connection with an LLM or another function included on a compute device. Disclosed embodiments provide conversion of low amplitude voice detection to text. The low amplitude voice detection can be accomplished in noisy environments. Disclosed embodiments can enable a full natural language input, enabling conversational AI.

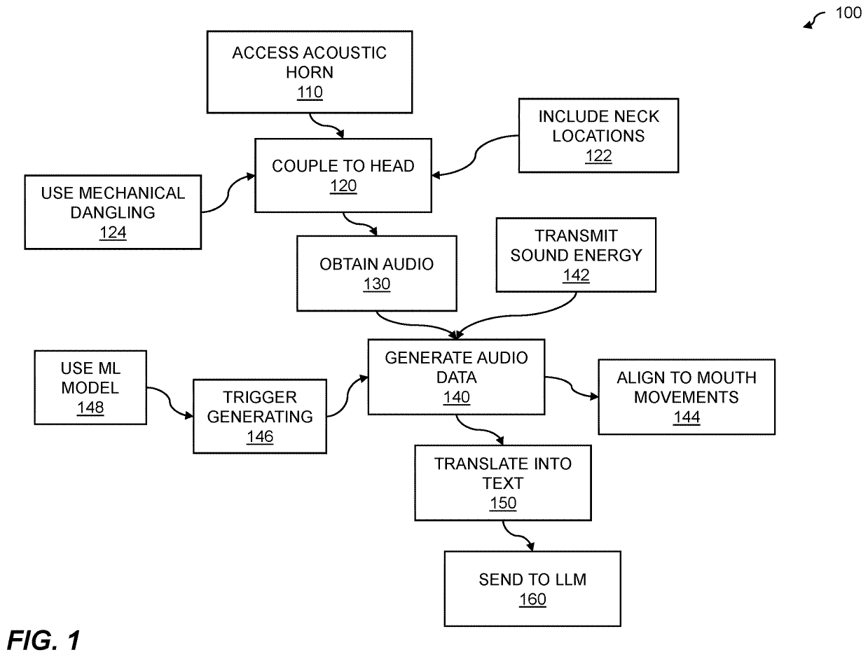

FIG. 1 is a flow diagram for conversion of low amplitude voice detection to text. The flow 100 includes accessing an acoustic horn 110. Embodiments include accessing an acoustic horn, wherein the acoustic horn is coupled to a head of a user. An acoustic horn can be a device which can capture audio signals. The acoustic horn can provide other functions such as amplifying the audio signals, controlling directionality of the audio signals, improving signal clarity of the audio signals, etc. In embodiments, the acoustic horn comprises a stethoscope. The acoustic horn can collect data via one or more techniques including a piezoelectric film (which can be positioned on the horn's diaphragm), one or more micro-electro-mechanical-systems (MEMS) microphones, one or more other microphones, and so on. A signal processing module (described below) can be coupled to the acoustic horn. In some embodiments, the acoustic horn includes a piezoelectric film. In other embodiments, the acoustic horn includes a microphone transducer. In embodiments, the acoustic horn includes one or more impedance matching layers. The impedance matching layers can include a gradient chamber. The gradient chamber can enable efficient transmission of low-amplitude vibrations, minimize signal loss between different media, and so on. The acoustic horn can include a geometry. The geometry can be optimized for low amplitude speech. The geometry can include one or more impedance matching layers. The optimization can include a location on the human body. For example, optimizations can be made to the acoustic horn for capturing low amplitude audio from a user's throat, side of the throat, etc. The same or different optimizations can be performed to capture audio from different locations on the user's head such as the temple, behind the ears, and so on.

The flow 100 includes coupling the acoustic horn to a user's head 120. The acoustic horn can be coupled to a user's head in any number of positions including either side of the neck, around either temple, on either cheek, behind either ear, and so on. The user's head can include neck locations 122. Although anatomically, the head and neck are considered separate, for speech and hearing purposes, the neck and head are inextricably linked, and the neck, throat, trachea, etc. are considered to be part of the head herein, which is consistent with common usage. Furthermore, the coupling to a user's head can comprise using mechanical dangling 124 of the acoustic horn from a chain, necklace, or equivalent, such that the head/neck provides mechanical support, but the acoustic horn can land in a position below the head/neck on the user's upper chest or even upper back. In embodiments, the coupling to the head of the user is located on a neck portion of the head. In embodiments, the coupling to the head of the user comprises a mechanical dangling of the acoustic horn from the head of the user. In other embodiments, the acoustic horn is included in a personal accessory. For example, the acoustic horn can be included in glasses, headphones, necklaces, and so on. This can accomplish the coupling while wearing the accessory.

The flow 100 includes obtaining audio 130. Embodiments include obtaining, by the acoustic horn, audio from a whispered voice of the user, wherein the whispered voice includes one or more words. The user can speak softly while the acoustic horn can capture the user's speech. The whispered voice can include speech at a low volume, a whisper, a mutter, speaking under the user's breath, and so on. The whispered voice can include minimal vocal cord vibration. The volume of the speech can be low. The volume of speech can be barely noticeable or undetectable to another person in proximity to the user. The whispered voice can lack clear phonetic structure. The one or more words can comprise a command, a series of commands, a sentence, a paragraph, a conversation, and so on. In a usage example, a user can interact, via the acoustic horn, with an LLM with full natural language input. Disclosed embodiments can enable full conversational AI with low volume speech.

The flow 100 includes generating audio data 140. Embodiments include generating audio data, by a signal processing module, wherein the audio data is based on the whispered voice that was obtained. As described above, the acoustic horn obtains audio. In embodiments, the acoustic horn transmits sound energy 142 from the user to the signal processing module. The transmission can be based on one or more devices such as a piezo film, a MEMS microphone, and so on. The transmission can be based on impedance matching using disclosed techniques. The signal processing module can comprise hardware, software, or a combination. The signal processing module can comprise a digital signal processing (DSP) pipeline, described later. The signal processing module can acquire the audio data from one or more sources; pre-amplify and/or amplify the audio data; perform functions such as filtering, spatial mapping, or beamforming; fuse one or more audio sources; and so on. The signal processing module can perform high-impendence preamplification for audio inputs from the acoustic horn such as a piezoelectric film, one or more MEMS microphones, and so on. The signal processing can transform audio input from the acoustic horn to provide intelligible speech. The intelligible speech can be sent to an automatic speech recognition (ASR) model (explained below). The signal processing module can comprise a listening device.

The flow 100 includes aligning to mouth movements 144. Further improvements can be made to enhance the quality of speech before passing it on to the ASR. Recall that a user's whisper speech can be captured by the acoustic horn. Additional devices can be used to supplement the capturing and processing of the speech. For example, an oral sensor interface worn by a user can also collect speech data. The oral sensor can include one or more of a tongue position sensor, an inertial measurement unit, a barometric pressure transducer, and so on. Additional speech data from the oral sensor interface can be used to help refine the audio data generated by the signal processing module. For example, the oral sensor can be used to align words spoken by the user with mouth movements. This can improve the sensitivity and accuracy of understanding especially low volume speech by the user. Embodiments include aligning the one or more words that were obtained with one or more mouth movements of the user. In some embodiments, the mouth movements are sensed by an oral sensor interface. In embodiments, the oral sensor interface comprises a mouthpad.

The flow 100 includes triggering the generating 146 of the audio data. The triggering can enable discrimination between speech, including whisper speech, and general background noise. The triggering can ensure that only actual speech is translated into text, described below, which can prevent “garbage” from being translated. In addition, it can enable power savings and extend battery life by turning off or quiescing any downstream electronics. The triggering can be performed using a trained machine learning model 148, which is described in more detail below. The machine learning model can comprise a neural network and can be trained for differentiating speech from noise using various labeled audio samples. Embodiments include assigning, by a machine learning model, a modality of the triggering sound. The modality can comprise a whisper, an audio voice, a gesture, a sound, a wake word, and so on. In some embodiments, the machine learning model comprises a logistic regression. A logistic regression can be trained on labeled audio features, such as spectral coefficients, temporal energy profiles, or phase alignment values, to learn weights that separate classes of input sounds. During training, the model can adjust its coefficients to minimize classification errors, thereby fine-tuning the decision boundary between modalities such as whisper speech and normal speech. Logistic regression models can provide efficient, low-power classification suitable for the signal processing device disclosed herein. Moreover, the logistic regression can be used alone or in combination with other models, providing a lightweight modality assignment mechanism that can be retrained and/or updated as new audio samples are collected. In some embodiments, the logistic regression performs speech classification. In some embodiments, the modality that was determined comprises a whisper. In further embodiments, the whisper enables conversational AI. Detecting whisper speech can activate downstream processing (explained below). Further, detecting whisper speech can replace a conventional wake word (such as “Hey Siri®”) or touch to wake a compute device (such as a smartphone). The detection can trigger conversational-AI capture and can be based on the captured signal's frequency signature and sensor fusion.

In other embodiments, the modality that was determined comprises audio speech. The audio speech can include speech in a normal (non-whisper) volume, such as conversational speech or raised speech. In some implementations, the modality assignment can distinguish audio speech from other modalities such as whisper speech, acoustic gestures, or mechanical gestures. The determination of audio speech can be based on spectral and/or temporal features characteristic of voiced speech, including harmonic structure and/or energy distribution across low-frequency bands. In some embodiments, the modality that was determined comprises a gesture. In further embodiments, the gesture comprises an acoustic gesture. In a usage example, the machine learning model detects a click of the tongue (an example of an acoustic, or audio, gesture). The click can initiate a conversation with an LLM, enter a transcription mode, etc. In one or more embodiments, the gesture comprises a mechanical gesture. In another usage example, a mechanical gesture such a single-tap, a double-tap, dragging a finger across the device, etc. can serve as a wake word sent to a coupled compute device. Any number of audio or mechanical gestures can be implemented.

In some embodiments, one or more classification weights within the logistic regression are based on an RMS value of the triggering sound. In some implementations, one or more classification weights within the logistic regression model can be derived from a root-mean-square (RMS) value of the triggering sound. The RMS value provides a measure of the average energy or amplitude of the audio signal, which can serve as a discriminative feature for distinguishing whisper speech from background noise or other modalities. During training, the logistic regression model can incorporate RMS values as input features, adjusting its coefficients to reflect the correlation between signal energy and modality classification. In operation, the RMS-based weights can bias the model toward recognizing low-energy signals as whisper speech, while higher-energy signals may be classified as normal speech or environmental sounds. In some embodiments, one or more classification weights within the logistic regression are based on a flatness of a frequency response of a signal captured by the acoustic horn. In some implementations, one or more classification weights within the logistic regression model can be based on a measure of spectral flatness of the frequency response of the signal captured by the primary and reference microphones. Spectral flatness can provide an indication of how noise-like or tone-like a signal is, with random background noise typically exhibiting a flatter frequency response than whisper speech or normal vocal speech. During training, the logistic regression model can incorporate spectral flatness values as input features, adjusting its coefficients to reflect the correlation between flatter responses and noise classification, and less flat responses and speech classification. In operation, the flatness-based weights can bias the model toward recognizing signals with structured harmonic content as intentional speech, while signals with flatter spectral profiles are classified as background noise.

In some embodiments, one or more classification weights within the logistic regression are based on a ratio of high frequency power to low frequency power. In some implementations, one or more classification weights within the logistic regression model can be based on a ratio of high-frequency power to low-frequency power of the triggering sound. This ratio provides a discriminative feature because whisper speech typically exhibits greater relative energy in higher frequency bands compared to normal vocal speech, which contains stronger low-frequency harmonic components. During training, the logistic regression model can incorporate the high-to-low frequency power ratio as an input feature, adjusting its coefficients to reflect the correlation between elevated high-frequency energy and whisper classification. In operation, signals with higher ratios can be weighted toward whisper speech detection, while signals with lower ratios can be weighted toward normal speech or background noise.

As described above, the modality can include whisper speech, normal speech, acoustic gestures, mechanical gestures, and so on. A whisper speech modality may be assigned in response to detecting speech exhibiting whisper-specific characteristics, such as reduced sound level and distinctive frequency envelope. Similarly, a normal speech modality may be assigned in response to detecting speech exhibiting characteristics consistent with conversational vocal intensity and spectral distribution. An acoustic gesture modality may be assigned in response to detecting non-verbal audio generated by the user, such as clicks, breaths, and/or other intentional sounds produced through the vocal tract. A mechanical gesture modality may be assigned in response to detecting non-verbal audio not produced by the vocal passage of the user, such as taps, scrapes, and/or other physical interactions with the signal processing module. Additional modalities may be defined in other implementations to accommodate further categories of input. In some embodiments, the assigning is based on an amplitude of the primary microphone, wherein the amplitude of the primary microphone associated with the triggering sound that was detected is higher than an amplitude of the reference microphone of the triggering sound that was detected. For example, if the device is worn on a shirt, the primary microphone will be louder than the reference microphone. In embodiments, the generating the audio is triggered using a machine learning model. In embodiments, the machine learning model performs a logistic regression.

The flow 100 includes translating into text 150. Embodiments include translating, by an automatic speech recognition (ASR) model, the audio data into text, wherein the text corresponds to the one or more words. An ASR model can convert spoken language into text. The ASR model can be tuned for low volume audio data such as described in disclosed embodiments. The ASR model can identify phonetic units, sound patterns, etc. The ASR can be based on Mel-frequency cepstral coefficients. The ASR model can convert any language to text. Thus, the user can whisper in any language to generate text. For example, the user can speak in English, Spanish, French, etc. As described above, the one or more words can comprise one or more commands, sentences, paragraphs, parts of natural speech, and so on. The signal processing module and the ASR can operate on a separate compute device coupled to a user's compute device, a mobile device, and so on. The signal processing module and the ASR can operate directly on the user's compute device, mobile device, etc.

The flow 100 includes sending to an LLM 160. Embodiments include sending, by the ASR model, the one or more words to a large language model (LLM). Once the words, sentences, phrases, utterances, etc. that the user whispered are translated into text by the ASR, that text can be sent to an LLM. In some implementations, the LLM operates in the cloud. In such cases, the sending can be based on a compute device, a mobile device, and so on. The sending can be based on technology such as Bluetooth®, WiFi, 4G LTE, 5G, Ethernet, and so on. In other cases, the LLM can reside on the compute device, mobile device, etc. which can also run the signal processing module and the ASR model. The sending can accomplish different purposes. In some implementations, the ASR can send the text to an LLM to improve the accuracy of the text. In other implementations, the text is sent to the same or a different LLM and used as a basis for one or more inferences. Other machine learning models can be used. The LLM can then send data back to the compute device, mobile device, etc. That device can then send the response to the user via a screen, text, alert, audio, etc. The user can wear headphones, or another earpiece which allows audio communication. Thus, the user can speak to the LLM in a whisper and obtain audio answers. Disclosed embodiments can enable full conversational AI with low volume speech. In embodiments, the acoustic horn, the signal processing module, and the ASR model comprise a speech sensor peripheral input device. The speech sensor peripheral input device can be coupled to any compute device. Thus, the sensor peripheral input device can enable low amplitude voice conversations with an LLM with various compute devices such as mobile phones, tablets, laptops, smartwatches, and so on.

Various steps in the flow 100 may be changed in order, repeated, omitted, or the like without departing from the disclosed concepts. Various embodiments of the flow 100, or portions thereof, can be included in a computer program product embodied in a non-transitory computer readable medium that includes code executable by one or more processors.

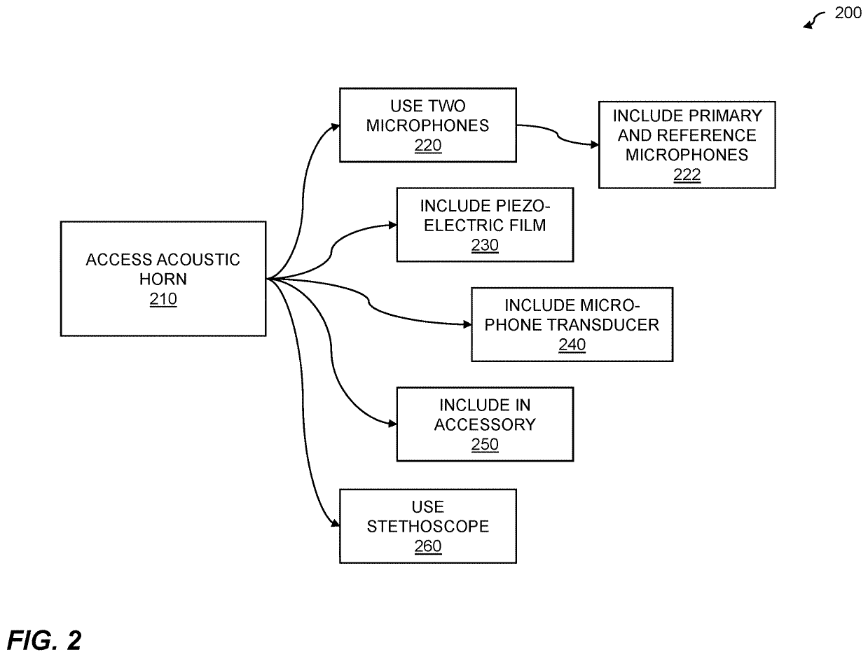

FIG. 2 is a flow diagram for acoustic horn constituency. Various acoustic horn constituencies can enable conversion of low amplitude voice detection to text. Various acoustic horn constituencies can be used singularly or in combination to provide optimal low-amplitude conversion. An acoustic horn is accessed. The acoustic horn is coupled to a head of a user. Audio from a whispered voice of the user is obtained. The whispered voice includes one or more words. The words can comprise a full natural language input. A signal processing module generates audio data. The audio data is based on the whispered voice that was obtained. The audio data is translated, by an automatic speech recognition (ASR) model, into text. The text corresponds to the one or more words. The ASR model can send the text to a large language model (LLM).

The flow 200 includes accessing an acoustic horn 210, as described above and throughout. The acoustic horn can include using two microphones 220. The two microphones can include a primary microphone and a reference microphone 222. The primary microphone can be oriented toward the user's mouth, and it can be configured to capture both near-field speech and far-field ambient sounds. The reference microphone can be oriented to preferentially sense chest-coupled vibrations and can be substantially acoustically shielded from environmental noise. In some embodiments, a triggering sound is detected, by the two microphones, from a mouth of a user. In other embodiments, the triggering sound is detected, by the two microphones, from a chest of a user. When a genuine voice utterance occurs, correlated signal components can be observed across both microphones, with the chest-coupled reference channel providing a distinctive low-frequency signature aligned in time with the mouth-directed signal. In contrast, background noise sources, such as conversations in the room or environmental sounds, predominantly register only at the primary microphone, with little or no corresponding activity at the reference microphone. In some embodiments, the determining is based on a voice activity detector (VAD) within the signal processing module. The VAD can include applying correlation analysis, energy thresholding, spectral comparison between the two channels, machine learning, etc. Disclosed implementations can discriminate user speech from ambient noise and thereby identify a triggering sound with improved reliability. In some other embodiments, the determining is based on alignment of the triggering sound with detected words from within the background noise. Determining that a triggering sound is speech can include aligning the detected acoustic features of the triggering sound with candidate word segments present in the background noise. Alignment can be performed by comparing temporal and spectral patterns of the triggering sound against stored phonetic models and/or reference word boundaries. If the triggering sound exhibits sufficient correspondence with a recognized word structure, disclosed implementations can classify the sound as intentional speech rather than ambient noise. For example, if a background consonant sound such as “s” is detected (such as is common in speech), disclosed embodiments can attempt to align the “s” sound with the triggering sound that was detected to ignore the background sound.

The acoustic horn can include a piezoelectric film 230, which can be positioned on the horn's diaphragm. The acoustic horn can include a microphone transducer 240. The three main types of microphone transducers, which convert sound waves into electrical signals, are known as dynamic, condenser, and ribbon transducers. For example, a dynamic microphone is the most basic type, and usually the least expensive, but it may not perform the best for all low amplitude speech detection scenarios. In embodiments, the acoustic horn includes one or more impedance matching layers. The impedance matching layers can include a gradient chamber. The gradient chamber can enable efficient transmission of low-amplitude vibrations, minimize signal loss between different media, and so on. The acoustic horn can include a geometry. The geometry can be optimized for low amplitude speech. The geometry can include one or more impedance matching layers. The optimization can include a location on the human body. For example, optimizations can be made to the acoustic horn for capturing low amplitude audio from a user's throat, side of the throat, etc. The same or different optimizations can be performed to capture audio from different locations on the user's head such as the temple, behind the ears, and so on. The acoustic horn can be included in or on a personal accessory 250, such as headphones or glasses. The coupling can occur on one or more places on the user's head. The acoustic horn can use a commercially available stethoscope 260, which provides un-transduced sound, but is readily obtainable and well known. It can also be incorporated into a personal accessory or even in clothing coupled to the head of a user. In embodiments, the acoustic horn includes two microphones. In embodiments, the two microphones comprise a primary microphone and a reference microphone.

Various steps in the flow 200 may be changed in order, repeated, omitted, or the like without departing from the disclosed concepts. Various embodiments of the flow 200, or portions thereof, can be included in a computer program product embodied in a non-transitory computer readable medium that includes code executable by one or more processors.

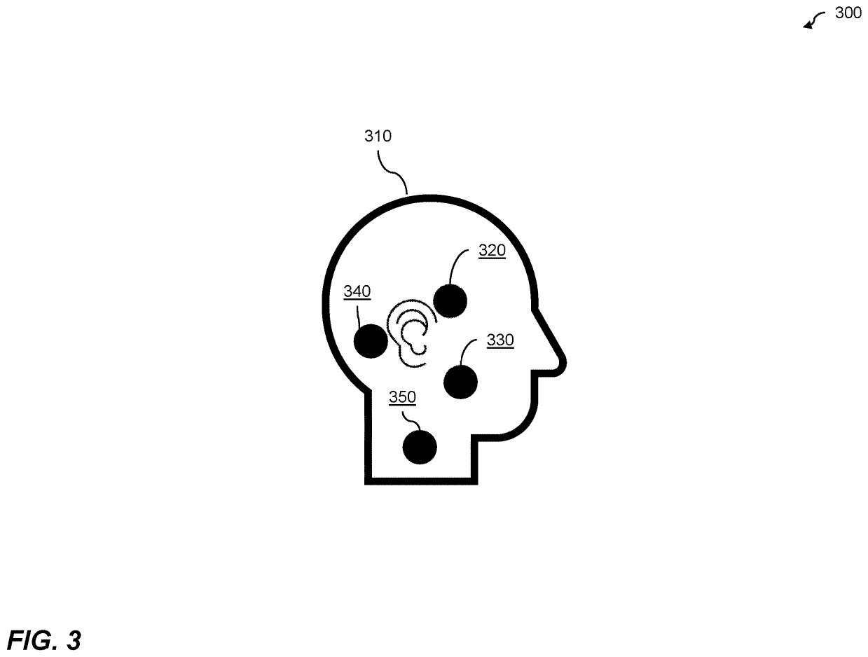

FIG. 3 is an example of placing an acoustic horn on a user. An acoustic horn can enable conversion of low amplitude voice detection to text. An acoustic horn is accessed. The acoustic horn is coupled to a head of a user. Audio from a whispered voice of the user is obtained. The whispered voice includes one or more words. The words can comprise a full natural language input. A signal processing module generates audio data. The audio data is based on the whispered voice that was obtained. The audio data is translated, by an automatic speech recognition (ASR) model, into text. The text corresponds to the one or more words. The ASR model can send the text to a large language model (LLM).

In the example 300, a user 310 is shown. The user can be any person speaking any language. The user can be in a noisy environment, a quiet environment, etc. To accomplish conversational AI with low amplitude speech, an acoustic horn is coupled to a head of the user. The coupling can be based on a personal accessory, such as headphones or glasses. The coupling can occur on one or more places on the user's head. As shown in example 300, the coupling can be accomplished near a user's temple 320. Sound waves generated by a user's whispered speech can be detected by the acoustic horn and the signal processing module and/or the ASR model can be tuned to generate text from speech from an area near the temple. The coupling can be accomplished on or near a user's cheek 330. Sound waves generated by a user's whispered speech can be detected by the acoustic horn on or near a user's cheek and the signal processing module and/or the ASR model can be tuned to generate text from speech from this area. The coupling can be accomplished behind a user's ear 340. Sound waves generated by a user's whispered speech can be detected by the acoustic horn behind a user's ear, and the signal processing module and/or the ASR model can be tuned to generate text from speech from this area. The coupling can be accomplished on a user's throat and/or neck 350. Sound waves generated by a user's whispered speech can be detected by the acoustic horn on the user's throat, which can include a side of the throat, a front of the throat, etc., and the signal processing module and/or the ASR model can be tuned to generate text from speech from this area. The coupling can be accomplished in other areas of the user's head.

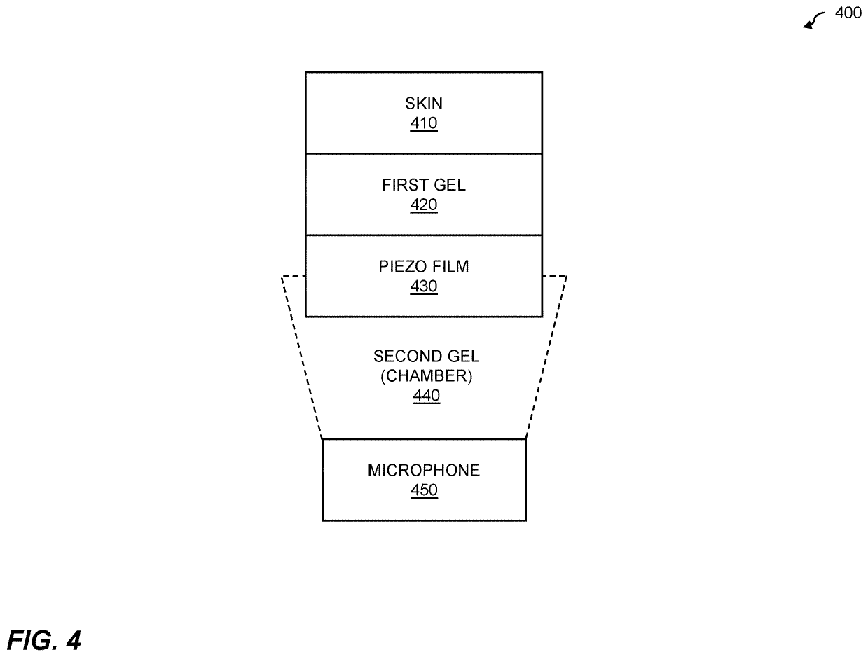

FIG. 4 is a diagram of an acoustic horn. An acoustic horn can enable conversion of low amplitude voice detection to text. An acoustic horn is accessed. The acoustic horn is coupled to a head of a user. Audio from a whispered voice of the user is obtained. The whispered voice includes one or more words. The words can comprise a full natural language input. A signal processing module generates audio data. The audio data is based on the whispered voice that was obtained. The audio data is translated, by an automatic speech recognition (ASR) model, into text. The text corresponds to the one or more words. The ASR model can send the text to a large language model (LLM).

As described previously, an acoustic horn can be a device which can capture audio signals. The acoustic horn can provide other functions such as amplification of the audio signals, controlling directionality of the audio signals, improving signal clarity of the audio signals, etc. In the diagram 400, the acoustic horn can contact the skin of a user 410. The skin can conduct faint audio signals from whispered speech of the user to the acoustic horn. As described above, the location of the skin making contact with the acoustic horn can include locations near the user's temple, near the user's cheek, behind the user's ear, on the user's throat, and so on. The acoustic horn can comprise a geometry tuned for impedance matching to efficiently send audio data from the user's skin to electronic functions such as the signal processing module, the ASR, and so on. The acoustic horn can include a first gel 420. The first gel can comprise an acoustic coupling system. The gel can comprise a low viscosity gel. The first gel can be between the user's skin and a piezo film 430, which can be a piezoelectric film. The piezo film can be positioned on the horn's diaphragm. The piezo film can generate electrical signal when vibration, such as from whispered speech collected from a user's skin, is applied. The bandwidth of collected audio sounds using this method can be wide, such as 200 Hz to 4 kHz, which can cover speech bandwidth. The piezo film can comprise a first method of audio collection.

The acoustic horn can include a second gel 440, which can be within an impedance gradient chamber. A microphone 450, such as a high-sensitivity MEMS microphone, can be positioned at the end of the chamber. The chamber can be designed to minimize signal loss between different media and can enable efficient transmission of low-amplitude vibrations. The chamber can enable microphone transduction. The microphone can provide a complementary detection method of the audio sounds generated from a user's whispered speech. Thus, the acoustic horn can comprise one or more methods of detecting whispered, or low-amplitude, speech by a user. The signal processing module can acquire data from multiple channels, which can include the piezo film and the microphone. The signal processing module can perform signal fusion from signals generated by the piezo film and the microphone.

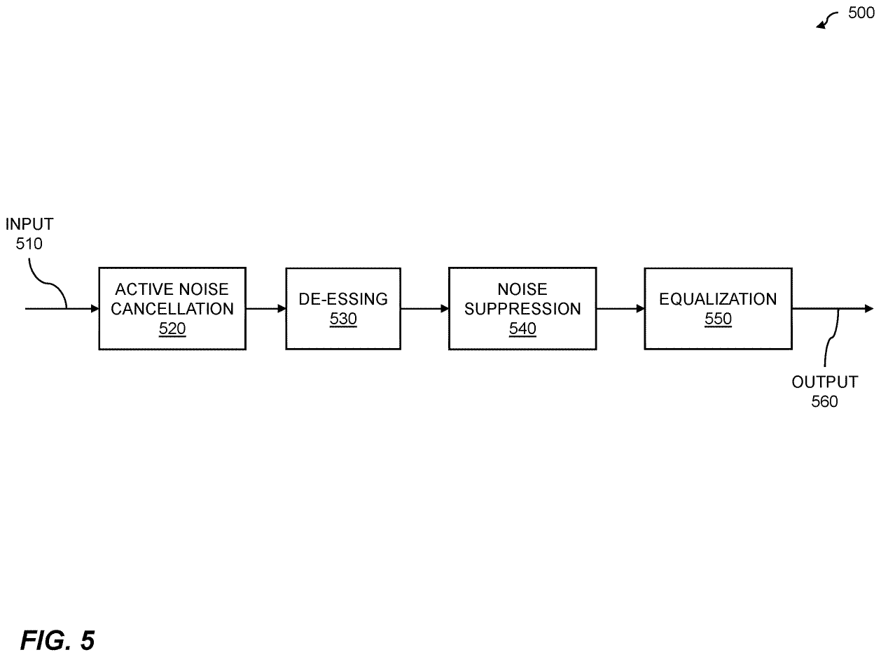

FIG. 5 is an infographic of signal processing using a digital signal processor (DSP) pipeline. A DSP pipeline can enable conversion of low amplitude voice detection to text. An acoustic horn is accessed. The acoustic horn is coupled to a head of a user. Audio from a whispered voice of the user is obtained. The whispered voice includes one or more words. The words can comprise a full natural language input. A signal processing module generates audio data. The audio data is based on the whispered voice that was obtained. The audio data is translated, by an automatic speech recognition (ASR) model, into text. The text corresponds to the one or more words. The ASR model can send the text to a large language model (LLM).

The infographic 500 shows an example DSP pipeline. The DSP pipeline can include a multistage pipeline that includes stages such as active noise cancellation, equalization, and others. In some embodiments, the DSP pipeline is within the processor of the signal processing module (described below). That is, the signal processing module can include hardware and software components necessary to run one or more DSP pipelines. In other implementations, the signal processing module can send input from the microphones to another device, such as a smartphone, on which the DSP pipelines can be implemented. The pipeline can be activated based on an assigned modality as described previously. In disclosed implementations, activation of a DSP pipeline may involve transitioning the processing chain from a low-power or idle state into an operational state in which it is prepared to accept continuous audio input and generate corresponding output. Activation can include loading filter coefficients into programmable stages, initializing gain parameters, and/or enabling clocking resources so that the pipeline operates in real time. In some implementations, activation may also involve allocating memory buffers, synchronizing input and output channels, and establishing control registers that govern adaptive behavior. By performing these initialization steps, the DSP pipeline can become fully configured to process incoming signals according to the assigned modality, thereby ensuring that whisper speech or other triggering sounds are captured and enhanced with minimal latency.

The infographic 500 includes an input 510 to the DSP pipeline and an output from the DSP pipeline 560. The input 510 would generally be sourced from the acoustic horn. The acoustic horn's output may be preconditioned before it is used as input to the DSP pipeline. The DSP pipeline output 560 can be used to trigger downstream processes as described throughout. In some embodiments, but not depicted here, the DSP pipeline includes beamforming the triggering sound, wherein the beamforming is based on minimum variance distortionless response (MVDR). In disclosed implementations, beamforming may be performed using the two microphones of the signal processing module, which can be spaced by a threshold distance, such as 10 mm, 25 mm, 60 mm, 100 mm, or more. Recall that the microphones can comprise a microphone array. Beamforming involves electronically adjusting the relative gain and phase of the microphone signals within the array to synthesize a directional sensitivity pattern, or lobe, that is oriented, for example, toward the mouth of a user. By steering the main lobe toward the expected source of the triggering sound, such as a verbal utterance coming from the mouth of a user, disclosed embodiments can increase sensitivity to near-field speech while attenuating off-axis noise sources. The chest-coupled reference microphone and the mouth-facing primary microphone can be combined in this process to reinforce correlated speech components and suppress uncorrelated ambient sounds. As a result, the beamformed output emphasizes the triggering sound and provides a cleaner signal for a subsequent enhancement and classification stage.

The DSP pipeline includes active noise cancellation 520. In disclosed implementations, the DSP pipeline may employ a variety of noise cancellation techniques, including adaptive filtering, spectral subtraction, Wiener filtering, and/or phase-based cancellation, each configured to attenuate ambient interference while preserving the low-energy spectral components characteristic of low amplitude or whisper speech. In some implementations, cancellation can be facilitated by structural modifications to the signal processing module. For example, the input port of one microphone in a dual-microphone configuration may be fitted with an air restrictor to limit the speed and amplitude of pressure waves entering that port, thereby reducing susceptibility to transient noise. The air restrictor can include open-cell foam. In certain implementations, both microphones are positioned against the chest within an external housing that functions as a low-pass acoustic filter. In disclosed implementations, an air restrictor can be disposed in a receiving path of the reference microphone and/or the primary microphone, which can enable attenuation. Noise entering from the front of the housing can be attenuated with open-cell foam and distributed evenly inside the housing, such that the resulting pressure waves strike each microphone substantially in phase. Because in-phase noise components can be readily suppressed through beamforming, this arrangement enables effective cancellation of environmental sounds. In disclosed embodiments, beamforming not only directs sensitivity toward the mouth of the user (i.e., the wearer of the signal processing module), but it can also enable cancellation of background noise that arrives at both microphones in phase. When environmental sounds strike the two microphones at substantially the same time and with similar amplitude, those signals are highly correlated. By combining the microphone outputs with appropriate weighting, disclosed implementations can subtract or otherwise suppress these common components. In contrast, speech, a whisper, a gesture, etc. originating from the mouth can reach the microphones with a measurable phase difference due to their spacing and orientation. The beamforming process of disclosed implementations can reinforce these out-of-phase speech components while cancelling the in-phase noise, thereby improving intelligibility of whispered utterances while cancelling background noise.

The DSP pipeline can include de-essing 530 of the triggering sound. De-essing can reduce one or more sibilant sounds within the triggering sound. In disclosed embodiments, de-essing can refer to reducing excessive sibilant energy that can be present in high-frequency sounds such as spoken words, whispering, gestures, etc. that include “s” or “sh.” These frequencies can dominate the spectral profile of whispered utterances. Moreover, since whisper speech lacks the strong harmonic structure of normal speech, sibilant components can appear disproportionately loud relative to the rest of the signal, which can impair intelligibility and listener comfort. De-essing may be performed by detecting spectral peaks in the sibilant frequency range (for example, 4-10 kHz) and applying dynamic attenuation through equalization, multiband compression, and/or adaptive filtering. In some implementations, the DSP pipeline can monitor energy levels in these bands and selectively reduce gain when sibilant transients exceed a threshold, while leaving lower-frequency whisper components unaffected. By controlling sibilance in this manner, disclosed implementations can produce a more balanced representation of whisper speech, improving clarity for downstream classification and transmission.

The DSP pipeline can include noise suppression 540 of the triggering sound. Noise suppression can be performed by a variety of filters, attenuators, spectral analyzers, and so on. Noise suppression can enable spurious background, environmental, or ambient sounds from being mistakenly analyzed as speech. The DSP pipeline can include equalizing the triggering sound 550, wherein the equalizing decreases one or more low frequency components of the triggering sound. The equalizing can include boosting and/or attenuating various frequency ranges. In disclosed implementations, equalization can be performed using common digital filtering and/or amplification techniques such as bandpass filtering, shelving filters, parametric equalizers, and multiband gain adjustment. These techniques can be applied to emphasize frequency ranges that carry intelligible components of whisper speech while reducing ranges that contribute to harshness or masking noise. In some implementations, the equalization parameters can be dynamically adjusted by the DSP pipeline based on detected signal characteristics, or it can be user-controlled through a settings block provided by a Bluetooth®-paired device such as a smartphone. In this way, a user can customize the output by selecting preferred tonal balance, boosting clarity bands, and/or attenuating sibilant regions, thereby tailoring the listening experience to individual preferences while maintaining intelligibility of whispered utterances. The equalizing can decrease one or more low frequency components. Low-frequency energy, such as rumble or chest-coupled vibrations, can mask the subtle spectral cues of whisper speech. By attenuating these components through high-pass filtering and/or dynamic low-shelf reduction, disclosed implementations can improve the understandability of whispered utterances and reduce listener fatigue.

In some embodiments, the equalizing includes individualized voice isolation. Individualized voice isolation can include adjusting frequency response parameters to emphasize spectral features unique to a particular user's voice while attenuating competing voices or background noise. For example, the signal processing module can store a profile of the user's vocal characteristics, such as dominant formant frequencies, harmonic structure, and/or timbre signatures, and apply equalization filters that boost those features relative to other sounds. In disclosed implementations, the individualized equalization can be derived from training data collected during first time setup, and/or dynamically updated based on ongoing use. By isolating the spectral components associated with the intended speaker, individualized voice isolation can improve intelligibility of whisper speech and reduce interference from nearby talkers or environmental sounds.

In some embodiments, the DSP pipeline includes envelope extraction (not shown) of the triggering sound, wherein the envelope extraction is based on one or more audio thresholds. In disclosed implementations, envelope extraction can serve as a final optional step to clean up the resulting signal prior to transcription or other downstream processing. After multiple stages of DSP enhancement, residual artifacts that manifest as low-level fluctuations or “chattering” noise superimposed on the whisper speech may remain. Envelope extraction can include applying simple thresholding and/or smoothing operations to the amplitude contour of the signal, thereby suppressing spurious variations that do not correspond to meaningful speech content. In some implementations, the DSP pipeline can monitor the extracted envelope and selectively attenuate components below a defined threshold, while preserving the primary energy profile of the whispered utterance. By performing this step, disclosed implementations can reduce artifact noise and deliver a cleaner, more intelligible signal for subsequent recognition and/or classification.

In some implementations, envelope processing can further include modifying attack, decay, sustain, and/or release characteristics of the whisper speech signal. For example, the DSP pipeline can attenuate sharp attack transients to reduce harshness, boost or extend rapid decays to preserve intelligibility, reinforce mid-level sustained energy to stabilize whispered utterances, and/or smooth release portions to minimize trailing artifact noise. By selectively shaping these envelope parameters, disclosed implementations can produce a more natural and intelligible representation of whisper speech, thereby improving recognition accuracy and listener comfort.

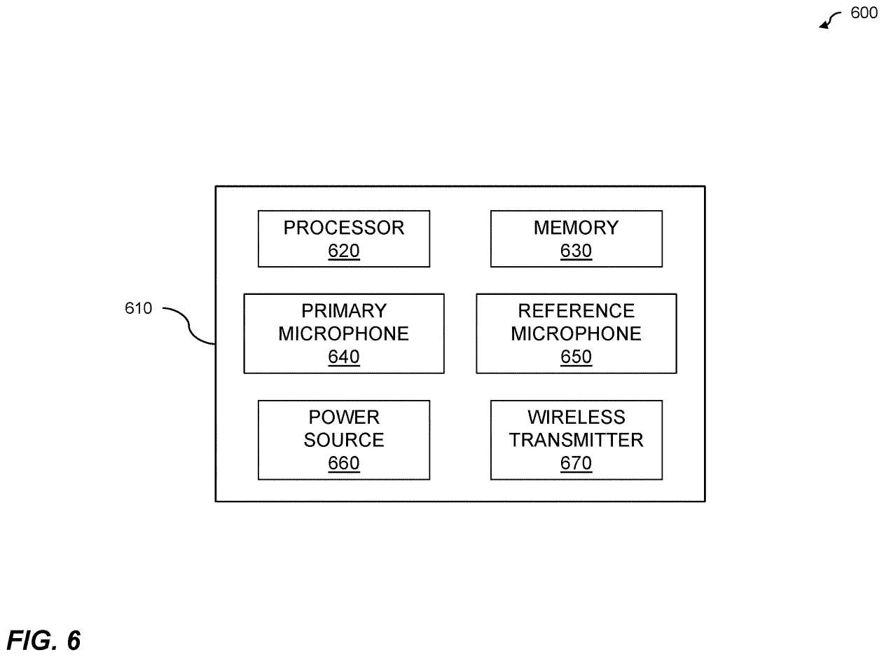

FIG. 6 is a block diagram for a dual-microphone signal processing module. A dual-microphone signal processing module can enable conversion of low amplitude voice detection to text. An acoustic horn is accessed. The acoustic horn is coupled to a head of a user. Audio from a whispered voice of the user is obtained. The whispered voice includes one or more words. The words can comprise a full natural language input. A signal processing module generates audio data. The audio data is based on the whispered voice that was obtained. The audio data is translated, by an automatic speech recognition (ASR) model, into text. The text corresponds to the one or more words. The ASR model can send the text to a large language model (LLM).

In the block diagram 600, a plurality of components is shown. The plurality of components shown in the block diagram 600 may be disposed within an integrated signal processing module (or device) 610. The signal processing device can be physically integrated with the two microphones that comprise the acoustic horn, as described above and throughout. Alternatively, it can be a discrete “electronics” package coupled to the two microphones of the acoustic horn. The signal processing module 610 can include a processor 620. The processor can comprise a low-power microcontroller suitable for compact wearable devices, such as an ARM® Cortex-M series processor, a DSP core which can be optimized for audio processing, an application-specific integrated circuit (ASIC), and/or other suitable controllers that are designed to operate with minimal energy consumption while providing sufficient computational capability to perform signal conditioning, compression, and wireless communication tasks.

The components can include a memory 630. The memory can include a combination of DRAM, SRAM, flash, and/or other volatile and non-volatile memory. In some implementations, the memory stores instructions that, when executed by the processor, implement one or more features of disclosed implementations, such as speech detection algorithms, noise suppression routines, and/or wireless transmission protocols. The memory may also store calibration data, user preferences, and/or firmware updates to support long-term operation of the device.

The components can include a primary microphone 640 and a reference microphone 650. In disclosed implementations, the primary microphone and the reference microphone can be physically separated by a distance threshold. In some embodiments, the two microphones include an acoustic microphone. In disclosed implementations, the microphones can include micro-electromechanical systems (MEMS) microphones, which provide a small size, low power consumption, and high sensitivity. MEMS microphones operate by converting acoustic pressure into electrical signals using a flexible diaphragm and capacitive sensing elements. In other implementations, the microphones can include electret condenser microphones (ECMs), piezoelectric microphones, laser microphones, and/or other miniature transducers suitable for detecting whisper-level speech. The choice of microphone type can be tailored to optimize sensitivity, frequency response, and durability in wearable applications. In some embodiments, the two microphones include a contact microphone. A contact microphone can detect mechanical vibrations conducted through a solid surface, such as the external housing of the signal processing module or the skin of the user, rather than relying solely on airborne acoustic waves. In disclosed implementations, the contact microphone can comprise a piezoelectric microphone element that generates an electrical signal in response to vibrational energy. By combining contact microphones with conventional air-coupled microphones, disclosed implementations can improve detection of whisper speech in noisy environments by isolating user-generated vibrations from background sounds.

The components can include a power source 660. In disclosed implementations, the power source can include a battery. The battery may be similar to those used in hearing aids, such as zinc-air coin cell batteries, lithium coin cells, or sealed rechargeable micro-batteries. In some implementations, the battery can be replaceable to allow convenient maintenance, while in other implementations the battery may be integrated as a rechargeable unit with wireless charging capability. The power source can be selected to balance compact size, long runtime, and safe operation in a wearable device.

The components can include a wireless transmitter 670. In disclosed implementations, the wireless transmitter can include a Bluetooth® and/or Bluetooth Low Energy® (BLE) module, enabling pairing with external electronic devices such as smartphones, tablet computers, or wearable computers (e.g., smartwatches). In other implementations, the transmitter may support alternative short-range communication protocols such as near-field communication (NFC) and/or proprietary low-power radio standards. The wireless transmitter can enable the apparatus to deliver audio data corresponding to whisper speech to external devices for further processing, such as speech-to-text conversion, amplification, and/or secure communication. The wireless transmitter can be a short-range wireless communication device. In embodiments, the signal processing module includes a short-range wireless communication device. Some embodiments include a short-range wireless communication device, wherein the short-range wireless communication device is coupled to the two microphones, and wherein the short-range wireless communication device sends, to a compute device, the whisper speech that was detected. In further implementations, the wireless transmitter 670 can comprise a wide-area transceiver such as an LTE, 5G, or other cellular radio module. Such a transceiver can enable the signal processing module to communicate directly with internet-connected devices or cloud services without requiring a nearby client device. This configuration can support real-time whisper conversations with remotely connected participants, secure transmission of audio data to cloud-based speech recognition engines, or direct interaction with networked voice-assistant platforms. As cellular modules become increasingly compact and cost-effective, disclosed implementations can leverage wide-area connectivity to broaden the usability of whisper speech detection beyond short-range environments.

In disclosed implementations, applications of the signal processing module extend beyond speech-to-text conversion. For example, the signal processing module can enable a user to conduct a phone conversation in public using only a whisper voice. In such implementations, the signal processing module captures whisper-level speech and transmits the audio data to a paired smartphone. The smartphone can employ additional processing to amplify the whisper speech, thereby making it intelligible to the remote caller. In some implementations, the smartphone may further cloak the whisper speech by overlaying or transforming the audio signal so that it resembles the user's normal speaking voice. This cloaking process can involve voice synthesis or timbre adjustment, such that the remote caller perceives the user as speaking in a conventional tone, even though the user is whispering. This feature can enable discreet communication in environments where privacy is desired, while maintaining natural conversational flow for the remote participant. In disclosed implementations, the signal processing module can cloak whisper speech by transforming the audio signal into a synthesized version of the user's normal speaking voice, thereby enabling a remote caller to perceive the user as speaking in a conventional tone.

In further implementations, the signal processing module can be configured to operate in rugged environments. The housing of the device may be designed to be waterproof or water-resistant, enabling reliable operation in rain, in humid conditions, or during outdoor activities. Waterproofing can be achieved through the use of sealed gaskets, conformal coatings on electronic components, and/or hydrophobic membranes covering microphone ports. In some implementations, the device may meet industry standards such as IPX7 or IPX8, allowing immersion in water for limited durations. Water resistance ensures that the signal processing module remains functional during daily wear, exercise, and/or exposure to environmental elements, thereby broadening its usability beyond controlled indoor settings. In disclosed implementations, the signal processing module can include a waterproof or water-resistant housing, wherein the housing comprises sealed gaskets, hydrophobic membranes, or conformal coatings to protect internal components from moisture, rain, or immersion.

In disclosed implementations, the signal processing module is capable of detecting an audio “gesture.” As used herein, an audio gesture refers to a sound produced by the user that is not a spoken word, but rather a deliberate acoustic signal intended to trigger a compute device function. Audio gestures can include acoustic gestures that comprise non-verbal sounds such as clicks, breaths, hums, and/or other controlled noises. For example, two quick gentle clicks produced by the user may be recognized by the signal processing module as a command to wake up a voice assistant on the paired smartphone. In another example, a long breath out may be interpreted as an instruction for the smartphone to read aloud the last incoming text message, start a dictation mode, and/or implement other suitable functions. Audio gestures can also include mechanical gestures, which can include finger taps and/or scrapes along a housing of a signal processing module. Thus, there are many combinations of audio gestures that can enable a hands-free, low-profile method of interacting with electronic devices without requiring spoken commands or manual input.

In some embodiments, the whisper enables conversational AI. The conversational AI can include discussions with an AI agent or chatbot for functions such as receiving directions to complete a task, obtaining information, receiving therapy, learning a foreign language, and a myriad of other tasks and activities. In disclosed implementations, the conversational AI can be configured to operate in real time, allowing whispered input to be processed and responded to immediately, even in noisy or crowded environments. The whispered interaction can provide a discreet and low-profile means of engaging with digital assistants, enabling users to access services without raising their voice or disturbing others nearby. The conversational AI can further include multimodal capabilities, such as combining whispered speech with gesture inputs and/or contextual data from paired devices, thereby enriching the dialogue and tailoring responses to the user's situation. In some implementations, the conversational AI can adapt to the user's preferences over time, learning individualized vocabulary, speech patterns, or whisper characteristics to improve recognition accuracy and personalization. In disclosed implementations, the conversational AI can support domain-specific modes that optimize recognition and interaction for particular subject areas. For example, in a medical guidance mode, the signal processing module can be fine-tuned to detect specialized terminology such as “hemoglobin” or “platelet” even when whispered, thereby improving accuracy of medical dialogue. In an educational tutoring mode, the system can emphasize recognition of subject-specific vocabulary such as “derivative” or “photosynthesis.” In enterprise task management mode, the system can prioritize workplace terminology such as “deliverable” or “compliance.” By enabling whispered conversational AI, disclosed embodiments provide enhanced privacy, reduced listener fatigue, and expanded opportunities for natural human-computer interaction across diverse applications.

In disclosed implementations, the audio gesture concept can be implemented through pattern recognition algorithms executed by the processor of the signal processing module and/or the paired smartphone. These algorithms can distinguish between intentional audio gestures and background noise, ensuring reliable activation of desired functions. In some implementations, the signal processing module may store a library of predefined audio gestures, while in other implementations the user may customize gestures to suit personal preferences. The use of audio gestures expands the interaction modalities available to the user, enabling control of electronic devices in situations where speech commands may be impractical or undesirable, such as in quiet public spaces or during physical activity. In disclosed implementations, the signal processing module can recognize audio gestures, wherein an audio gesture comprises a non-verbal sound intentionally produced by the user, such as clicks, hums, or breaths, and wherein recognition of the audio gesture triggers a corresponding compute device function. In disclosed implementations, the signal processing module can interpret two quick clicks as a command to activate a voice assistant on a paired smartphone. In disclosed implementations, the signal processing module can interpret a long exhale as a command to instruct the smartphone to read aloud the last incoming text message. The sound from the mouth of the user can comprise an utterance, wherein the utterance represents a gesture. In some implementations, the interpretation of audio gestures can be performed on the paired compute device.

In certain implementations, audio gestures may be combined with whisper speech to create hybrid control schemes. For example, a user may whisper a command word following an audio gesture, allowing the device to interpret both the gesture and the speech for enhanced accuracy. This multimodal approach provides flexibility and robustness in user interaction, ensuring that the signal processing module can adapt to diverse environments and user needs. The introduction of audio gestures represents a novel extension of acoustic input beyond traditional speech recognition, offering new possibilities for intuitive and discreet device control. In disclosed implementations, the signal processing module can combine audio gestures with whisper speech commands, wherein the combination provides multimodal input for enhanced accuracy and flexibility in device control.

In some implementations, a number of audio samples may be temporarily stored in memory 630 and later transmitted to a connected device, such as a Bluetooth-paired smartphone. In the event the smartphone (or other paired device) is temporarily offline, the stored audio samples can be transferred to the smartphone when the smartphone is reconnected to the signal processing module. In disclosed implementations, the audio samples can be stored in circular buffers, or other suitable structures.

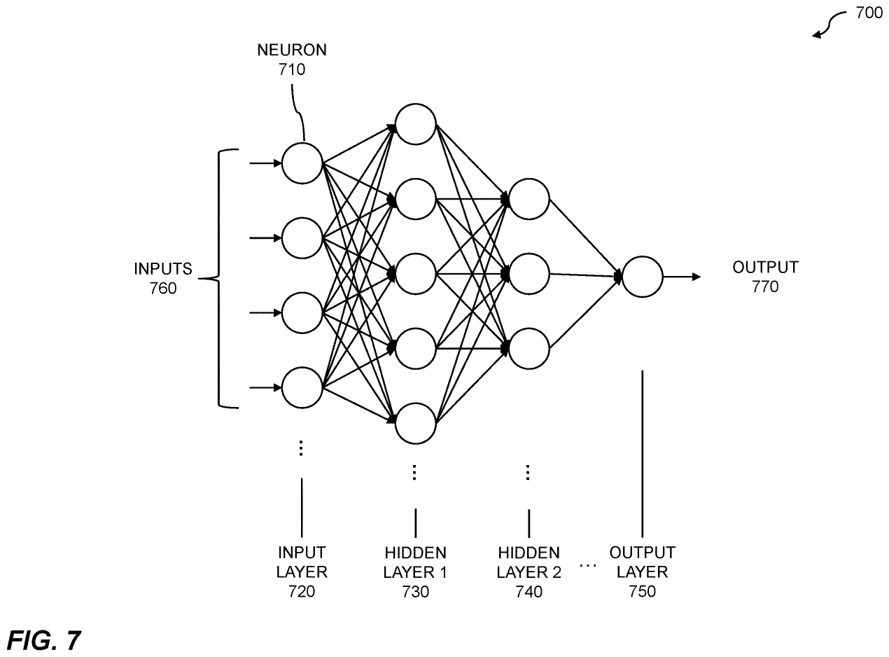

FIG. 7 is an example neural network. Various architectures can be used for a machine learning model, including the example neural network. The example neural network 700 includes a plurality of layers, where each layer is comprised of one or more neurons, such as neuron 710. The layers can be fully connected, where each neuron (or “node”) of a previous layer is coupled to each node of a subsequent layer, or can be partially connected. The neural network can comprise a deep learning network (i.e., which generally includes many hidden layers), a convolutional neural network (i.e., which generally includes a convolutional layer that can apply one or more filters across inputs), and so on. The neurons can perform calculations to determine a weighted sum of inputs 760. Each neuron can include a bias which can determine or alter the impact of the calculation. The neuron can include an activation function such as a sigmoid function, a rectified linear unit (ReLU) normalization function, a hyperbolic tangent (tanh) function, and so on. The activation function is used to ensure that the value calculated by the neuron remains between 0 and 1. Weights and biases can determine how much the value in the current neuron should affect other neurons in the next layer. The number of weights and biases can be referred to as a number of parameters in the model. A neural network can easily comprise one million parameters and 500,000 neurons, but smaller and larger model sizes are possible, based on available compute resources, expected battery life, desired result precision, and so on.

The neurons can be arranged into layers. The output of a neuron in one layer can be used to feed one or more neurons in next layer. When all neurons in a previous layer feed neurons in a following layer, the following layer can be referred to as a fully connected layer, or a dense layer. One or more layers can comprise fully connected layers. The various layers and connections between layers can form the basis of an inference operation by the neural network. The neural network can include an input layer 720 which collects external data as input to the model. The data collected and collated at the input layer can comprise scalar values, vector values, matrices, or higher-dimensional representations of data such as tensors. The neural network can comprise any number of hidden layers with varying numbers of nodes. The hidden layers can comprise any number of fully connected layers, convolutional layers, pooling layers, and so on. In the example 700, two hidden layers are shown, hidden layer 1 730 and hidden layer 2 740. In practice, there can be many hidden layers in the network. An output layer 750 can generate the desired output 770, once the network has been appropriately trained. Although only one node is shown in output layer 750, other output layer configurations are possible.

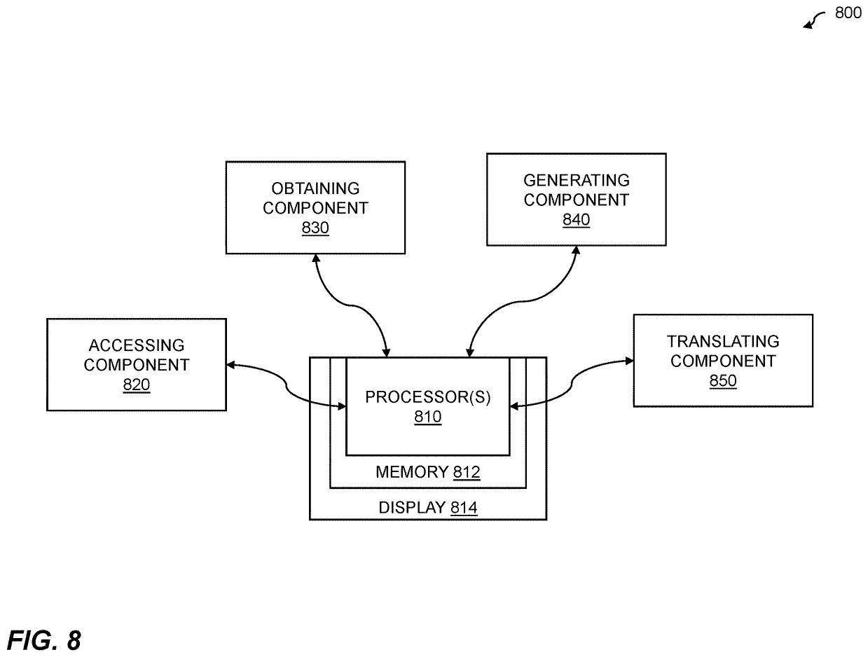

FIG. 8 is a system diagram for conversion of low amplitude voice detection to text. The system 800 can include one or more processors 810 and a memory 812 which stores instructions. The memory 812 is coupled to the one or more processors 810, wherein the one or more processors 810 can execute instructions stored in the memory 812. The memory 812 can be used for storing instructions, runtime libraries, audio data, signal processing routines, machine learning models, ASR models, error codes or error handling routines, and so on. Information such as audio data, text, and so on can be shown on a display 814 connected to the one or more processors 810. The display can comprise a television monitor, a projector, a computer monitor (including a laptop screen, a tablet screen, a netbook screen, and the like), a smartphone display, a mobile device, or another electronic display.

The system 800 can include a computer system for audio processing comprising: a memory which stores instructions; and one or more processors coupled to the memory, wherein the one or more processors, when executing the instructions which are stored, are configured to: access an acoustic horn, wherein the acoustic horn is coupled to a head of a user; obtain, by the acoustic horn, audio from a whispered voice of the user, wherein the whispered voice includes one or more words; generate audio data, by a signal processing module, wherein the audio data is based on the whispered voice that was obtained; and translate, by an automatic speech recognition (ASR) model, the audio data into text, wherein the text corresponds to the one or more words.

The system 800 can include a computer program product embodied in a non-transitory computer readable medium for audio processing, the computer program product comprising code which causes one or more processors to perform operations of: accessing an acoustic horn, wherein the acoustic horn is coupled to a head of a user; obtaining, by the acoustic horn, audio from a whispered voice of the user, wherein the whispered voice includes one or more words; generating audio data, by a signal processing module, wherein the audio data is based on the whispered voice that was obtained; and translating, by an automatic speech recognition (ASR) model, the audio data into text, wherein the text corresponds to the one or more words.

The system 800 includes an accessing component 820. The accessing component includes functions and instructions for accessing an acoustic horn, wherein the acoustic horn is coupled to a head of a user. Described above and throughout, the acoustic horn can collect data via one or more techniques and/or devices including a piezoelectric film (which can be positioned on the horn's diaphragm), one or more micro-electro-mechanical-systems (MEMS) microphones, one or more other microphones, and so on. In embodiments, the acoustic horn includes one or more impedance matching layers. The impedance matching layers can include a gradient chamber. The gradient chamber can enable efficient transmission of low-amplitude vibrations, minimize signal loss between different media, and so on. The acoustic horn can include a geometry. The geometry can be optimized for low amplitude speech. The acoustic horn can be coupled to a user's head in any number of positions including either side of the neck, around either temple, on either cheek, behind either ear, and so on. In some embodiments, the acoustic horn is included in a personal accessory. In a usage example, the acoustic horn can be included in glasses, headphones, and so on. In embodiments, the acoustic horn comprises a stethoscope.

The system 800 includes an obtaining component 830. The obtaining component includes functions and instructions for obtaining, by the acoustic horn, audio from a whispered voice of the user, wherein the whispered voice includes one or more words. The acoustic horn can capture the user's whispered voice. The whispered voice can include speech at a low volume, a whisper, a mutter, speaking under the user's breath, and so on. The whispered voice can include minimal vocal cord vibration. The volume of the speech can be low. The volume of speech can be barely noticeable or even undetectable to another person in proximity to the user. The whispered voice can lack clear phonetic structure. The one or more words can comprise a command, a series of commands, a sentence, a paragraph, a conversation, and so on.

The system 800 includes a generating component 840. The generating component includes functions and instructions for generating audio data, by a signal processing module, wherein the audio data is based on the whispered voice that was obtained. The signal processing module can comprise hardware, software, or a combination. The signal processing module can comprise a digital signal processing (DSP) function(s). The signal processing module can acquire the audio data from one or more sources; pre-amplify and/or amplify the audio data; perform functions such as filtering, spatial mapping, and beamforming; fuse one or more audio sources; and so on. The signal processing can transform audio input from the acoustic horn to provide intelligible speech.

The system 800 includes a translating component 850. The translating component includes functions and instructions for translating, by an automatic speech recognition (ASR) model, the audio data into text, wherein the text corresponds to the one or more words. As described previously, an ASR model can convert spoken language into text. The ASR model can be tuned for low volume audio data such as described in disclosed embodiments. The ASR model can identify phonetic units, sound patterns, etc. The ASR can be based on Mel-frequency cepstral coefficients. The ASR model can convert any language to text. Thus, the user can whisper in any language to generate text. The signal processing module and the ASR can operate on a separate compute device coupled to a user's compute device, a mobile device, and so on. The signal processing module and the ASR can operate directly on the user's compute device, mobile device, etc.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagram and flow diagram illustrations depict methods, apparatus, systems, and computer program products. The elements and combinations of elements in the block diagrams and flow diagrams show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general-purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are limited to neither conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM); an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript®, ActionScript®, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States, then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A processor-implemented method for audio processing comprising:
accessing an acoustic horn, wherein the acoustic horn is coupled to a head of a user;
obtaining, by the acoustic horn, audio from a whispered voice of the user, wherein the whispered voice includes one or more words;
generating audio data, by a signal processing module, wherein the audio data is based on the whispered voice that was obtained; and
translating, by an automatic speech recognition (ASR) model, the audio data into text, wherein the text corresponds to the one or more words.

2. The method of claim 1 further comprising sending, by the ASR model, the one or more words to a large language model (LLM).

3. The method of claim 1 wherein the acoustic horn transmits sound energy from the user to the signal processing module.

4. The method of claim 3 wherein the acoustic horn includes one or more impedance matching layers.

5. The method of claim 1 wherein the acoustic horn, the signal processing module, and the ASR model comprise a speech sensor peripheral input device.

6. The method of claim 1 further comprising aligning the one or more words that were obtained with one or more mouth movements of the user.

7. The method of claim 6 wherein the mouth movements are sensed by an oral sensor interface.

8. The method of claim 1 wherein the generating the audio is triggered using a machine learning model.

9. The method of claim 8 wherein the machine learning model comprises a logistic regression.

10. The method of claim 1 wherein the signal processing module includes a digital signal processing (DSP) pipeline.

11. The method of claim 10 wherein the DSP pipeline performs active noise cancellation.

12. The method of claim 1 wherein the coupling to the head of the user is located on a neck portion of the head.

13. The method of claim 1 wherein the coupling to the head of the user comprises a mechanical dangling of the acoustic horn from the head of the user.

14. The method of claim 1 wherein the acoustic horn includes a piezoelectric film.

15. The method of claim 1 wherein the acoustic horn includes a microphone transducer.

16. The method of claim 1 wherein the acoustic horn is included in a personal accessory.

17. The method of claim 1 wherein the acoustic horn comprises a stethoscope.

18. The method of claim 1 wherein the acoustic horn includes two microphones.

19. The method of claim 18 wherein the two microphones comprise a primary microphone and a reference microphone.

20. A computer program product embodied in a non-transitory computer readable medium for audio processing, the computer program product comprising code which causes one or more processors to perform operations of:
accessing an acoustic horn, wherein the acoustic horn is coupled to a head of a user;
obtaining, by the acoustic horn, audio from a whispered voice of the user, wherein the whispered voice includes one or more words;
generating audio data, by a signal processing module, wherein the audio data is based on the whispered voice that was obtained; and
translating, by an automatic speech recognition (ASR) model, the audio data into text, wherein the text corresponds to the one or more words.

21. A computer system for audio processing comprising:
a memory which stores instructions; and
one or more processors coupled to the memory, wherein the one or more processors, when executing the instructions which are stored, are configured to:
access an acoustic horn, wherein the acoustic horn is coupled to a head of a user;
obtain, by the acoustic horn, audio from a whispered voice of the user, wherein the whispered voice includes one or more words;
generate audio data, by a signal processing module, wherein the audio data is based on the whispered voice that was obtained; and
translate, by an automatic speech recognition (ASR) model, the audio data into text, wherein the text corresponds to the one or more words.
